(19) United States
(12) Patent Application Publication
(10) Pub. No.: US 2014/0103008 A1
(43) Pub. Date: Apr. 17, 2014

(54) METHOD OF FABRICATING TEST STRIP FOR MEASURING BIOLOGICAL FLUID

(71) Applicant: ICHIA TECHNOLOGIES, INC., Taoyuan (TW)

(72) Inventor: Ko-Ju CHEN, Taoyuan (TW)

(73) Assignee: ICHIA TECHNOLOGIES, INC., Taoyuan (TW)

(21) Appl. No.: 13/722,928

(22) Filed: Dec. 20, 2012

(30) Foreign Application Priority Data
Oct. 15, 2012 (TW) 101137883

(51) Int. Cl. A61B 5/15

(52) U.S. Cl. CPC A61B 5/150274
USPC 216/20

(57) ABSTRACT

The invention provides a method of fabricating test strip for measuring biological fluid. The method comprises steps of: preparing a metallic membrane and an insulating material; adhering the metallic membrane on the insulating material; photolithographing for the metallic membrane; etching the metallic membrane to form a circuit layer; providing a sensing reagent on the circuit layer to form a sensing reagent layer; and adhering an intermediate layer and a cover to the insulating material to cover the circuit layer and expose part of the circuit layer. The circuit layer is provided in an adhering manner to enhance the stability of resistance and the test accuracy.

This application is based on and claims the benefit of Taiwan Application No. 101137883 filed Oct. 15, 2012 the entire disclosure of which is incorporated by reference herein.

### BACKGROUND OF THE INVENTION

1. Field of the Invention

The present invention relates to a biological test strip, and more in particular, to a test strip for measuring biological blood.

2. Related Art of the Invention

A blood glucose test is one of blood test items. One of the current technologies is proposed including steps of: blood is instilled on a test strip, the blood is sensed by a sensing reagent, a signal is transmitted to a blood glucose meter by a metallic circuit of the test strip, and blood glucose concentration of the blood is measured by the blood glucose meter.

A conventional biosensor such as electrode type test strip includes a base body with a printed surface, an intermediate layer and a cover. The printed surface has a reactive membrane, two electrode membranes, a conductive electrode membrane and a reference electrode membrane. The conductive electrode membrane is disposed between two electrode membranes. The two electrode membranes connect with the reactive membrane at one end of each electrode membrane. The conductive electrode membrane connects to one of two electrode membranes at adjacent the reactive membrane. The reference electrode membrane is disposed and opposite to one of two electrode membranes at two sides of the printed surface. The reference electrode membrane extends to contact the reactive membrane at one end. The intermediate layer has an opening corresponding to the reactive membrane, and the cover has a test hole corresponding to the opening. The cover and the intermediate layer are disposed on the base body in sequence, and the intermediate layer is interposed between the base body and the cover and adhesive to all together.

The conductive layer (electrode) is fabricated by printing a conductive carbon powder and silver paste on the base body in a printing process. The conductive layer fabricated in a printing manner has a poor test accuracy due to a poor stability of resistance.

### SUMMARY OF THE INVENTION

It is an object of the invention to provide a method of fabricating test strip in an adhering manner to solve the above-described drawback of the conventional method. The circuit layer is provided in attaching manner to enhance the stability of resistance and the test accuracy.

In order to achieve the above-described object, the invention provides a method of fabricating test strips for measuring biological blood. The method comprises steps: preparing a metallic membrane and an insulating material; adhering the metallic membrane on the insulating material; photolithographing for the metallic membrane; etching the metallic membrane to form a. circuit layer connected to an electrode section; providing a sensing reagent on the electrode section to form a sensing reagent layer; adhering an intermediate layer to the insulating material to cover the circuit layer and expose part of the circuit layer, the intermediate layer having an opening to expose the sensing reagent layer and the electrode section; and adhering a cover to cover the intermediate layer and the insulating material and expose part of the circuit layer, the cover having two holes corresponding to the opening.

The metallic membrane may be conductive metallic material selected from the group consisting of palladium, nickel, platinum, tungsten, gold, titanium, copper, zinc, iridium, vanadium and zirconium

The insulating material may be paper, timber, Acrylonitrile-Butadiene-Styrene (ABS), polycarbonate (PC), polyethylene terephthalate (PET), polyimide (PI), thermoplastic elastomer or polylactic acid plastic.

A mask resist layer is formed on the metallic membrane, and exposed by a stepper to form an exposed film through the reaction of resist layer. Next, a development process is performed, and an unexposed resist layer is dissolved.

The exposed portion of the metallic membrane is etched in an etching process. Next, the residual resist is stripped, and a metallic circuit layer is formed on the insulating material.

The metallic circuit layer includes an electrical connector, a metallic circuit connecting to the electrical connector and an electrode section connecting to an end of the metallic circuit.

The amount and species of the sensing reagent are selected as required. The sensing reagent is applied on the electrode section to form a sensing reagent layer by spray marking.

The intermediate layer is adhered to the insulating material to cover the metallic circuit and the electrode section of the circuit layer and expose the electrical connector. The intermediate layer is shorter than the insulating material. The intermediate layer is of insulating material.

After the cover is covered, the test strip only exposes the electrical connector covered with the metallic film 1b. The cover is shorter than the insulating material. The cover is of insulating material.

The insulating material is cut to form a plurality of insulating sheets. Each of the insulating sheets has the circuit layer, the sensing reagent layer, the intermediate layer and the cover.

A metallic film is plated on the metallic circuit layer. The metallic film may be conductive metallic material selected from the group consisting of palladium, nickel, platinum, tungsten, gold, titanium, copper, zinc, iridium, vanadium and zirconium.

The resistance of the circuit layer has a range from 0.01Ω to 2k Ω. The thickness of the circuit layer has a range from 0.02 um to 30 um.

### DESCRIPTION OF THE DRAWINGS

FIG. 1 schematically shows a flow chart of fabricating test strips for measuring. biological fluid of an embodiment of the invention.

FIGS. 2-6 schematically show cross-sectional views of a test strip by a part of steps of fabricating process of an embodiment of the invention.

FIG. 7 is a top view of FIG. 6.

FIG. 8 schematically shows a cross-sectional view of a test strip after forming a metallic film on a metallic circuit layer of an embodiment of the invention.

FIGS. 9-12 schematically show top views of a test strip by a part of steps of fabricating process of an embodiment of the invention.

FIG. 13 schematically shows a cross-sectional view of FIG. 12.

### DETAILED DESCRIPTION OF THE INVENTION

The present invention will be apparent from the following detailed description, which proceeds with reference to the accompanying drawings. The accompanying drawings are not meant to be construed in a limiting sense, which are only for reference and explanation.

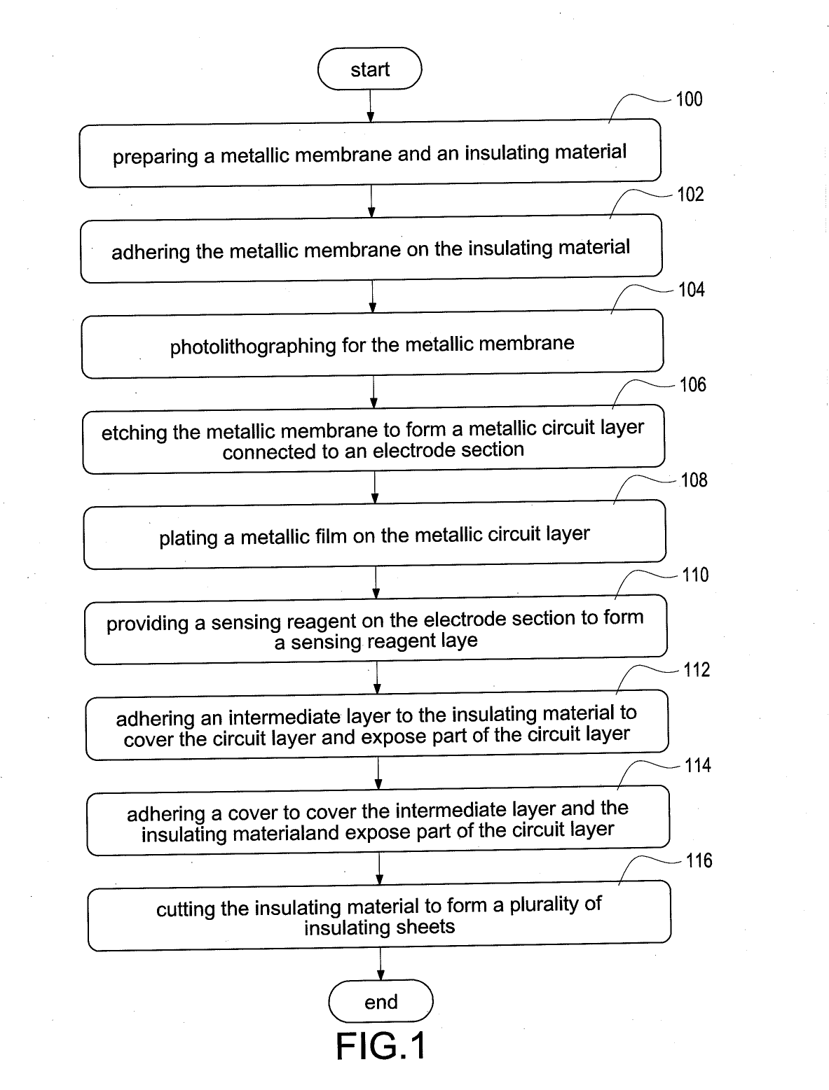

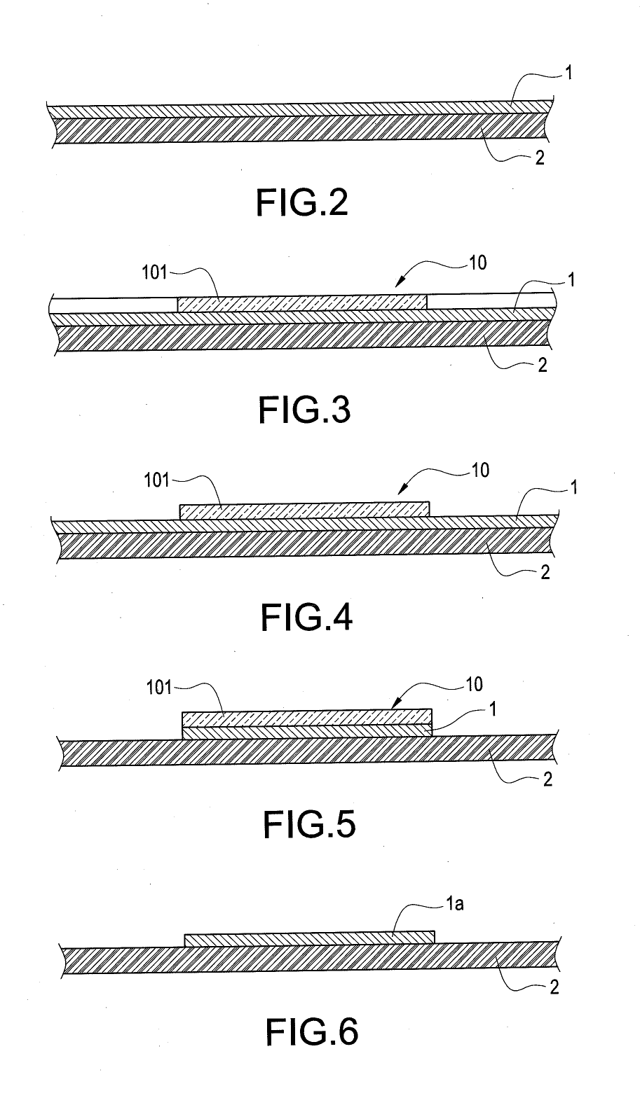

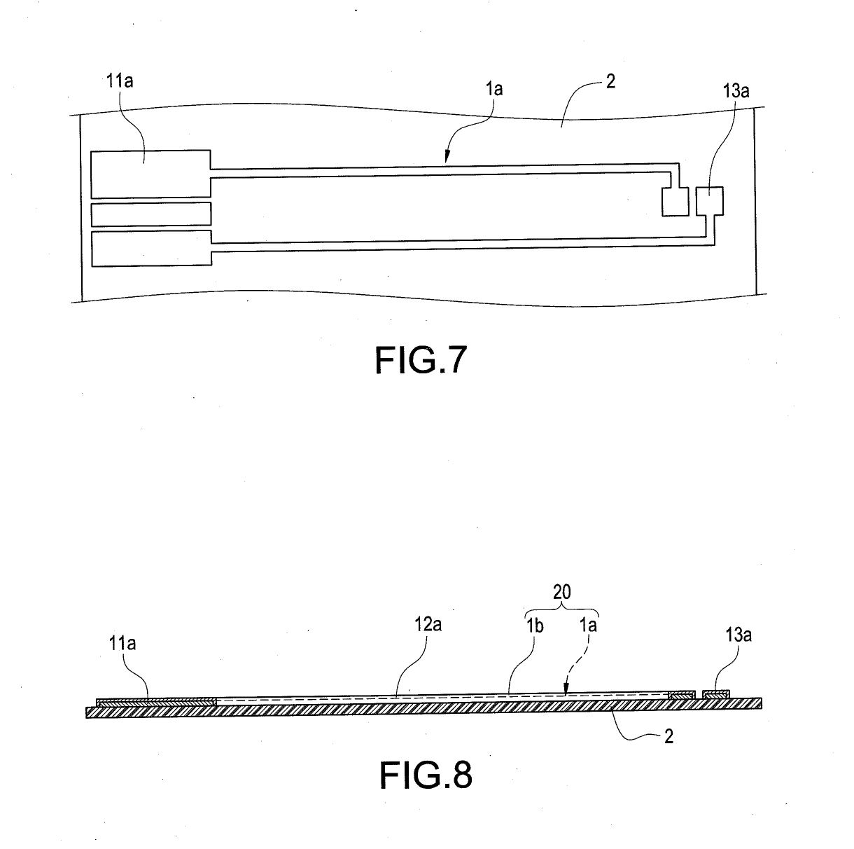

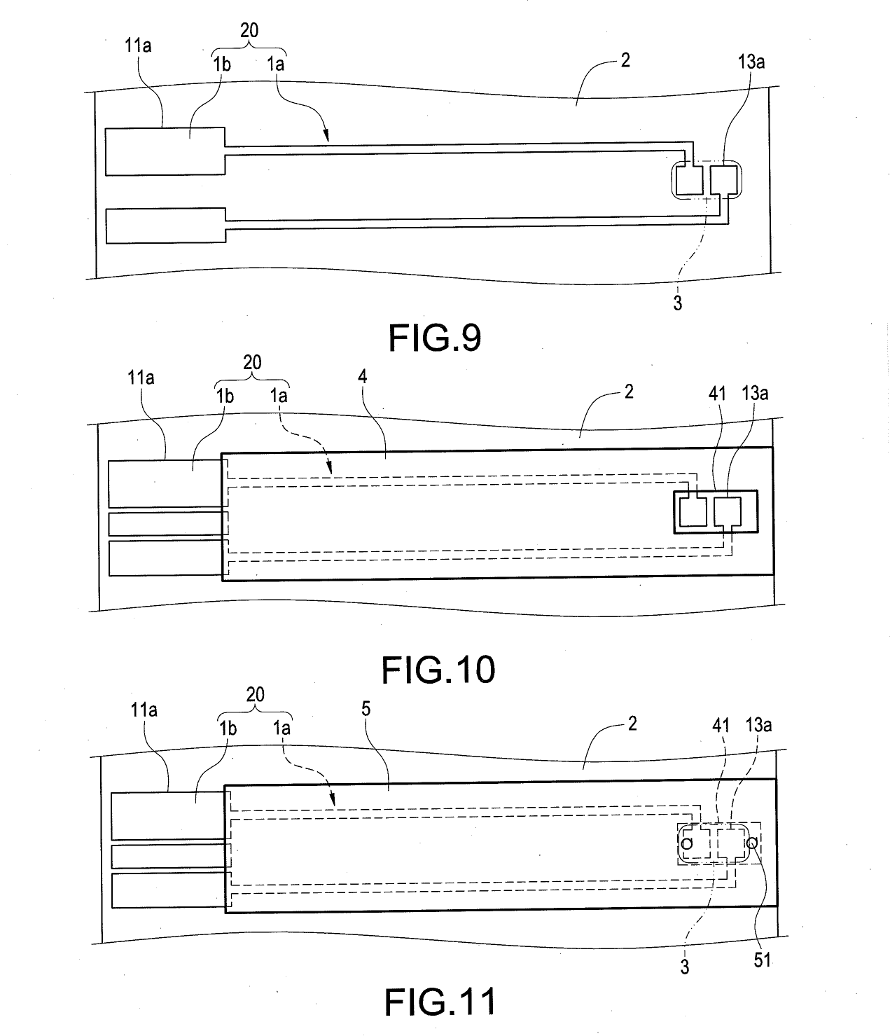

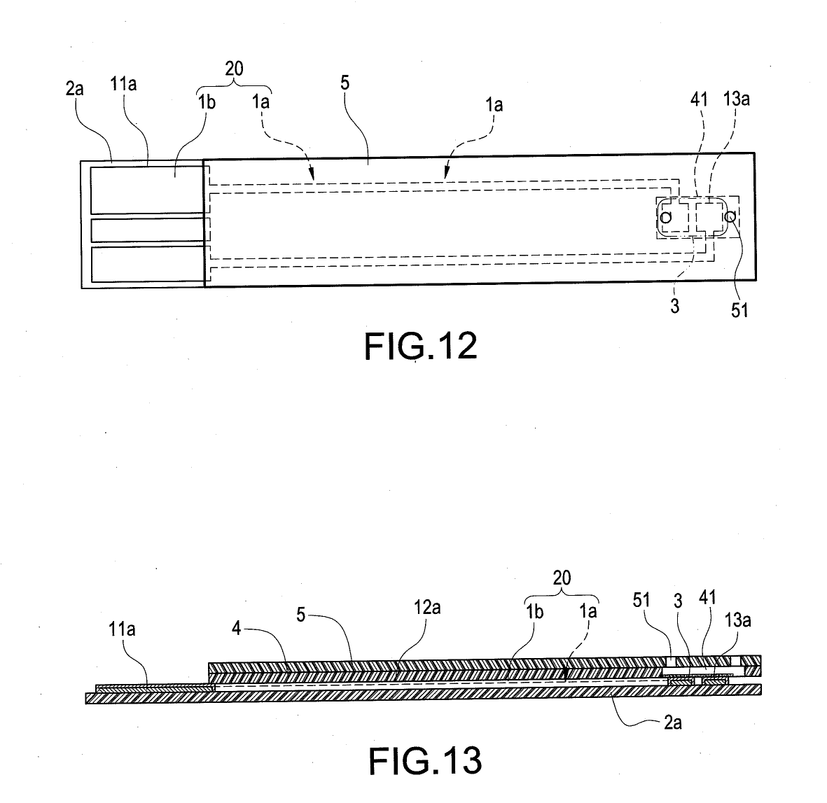

Please refer to FIG. 1. FIG. 1 schematically shows a flow chart of fabricating test strips for measuring biological fluid of an embodiment of the invention. Please also refer to FIGS. 2-13. FIGS. 2-6 schematically show cross-sectional views of a test strip by a part of steps of fabricating process of an embodiment of the invention. FIG. 7 is a top view of FIG. 6. FIG. 8 schematically shows a cross-sectional view of a test strip after forming a metallic film on a metallic circuit layer of an embodiment of the invention. FIGS. 9-12 schematically show top views of a test strip by a part of steps of fabricating process of an embodiment of the invention. FIG. 13 is a cross-sectional view of FIG. 12. As shown in drawings, the method of fabricating test strips for measuring biological fluid of the invention is provided. At first, in step 100, a metallic membrane 1 is prepared. In the embodiment, the metallic membrane 1 may be conductive metallic material selected from the group consisting of palladium, nickel, platinum, tungsten, gold, titanium, copper, zinc, iridium, vanadium and zirconium, as shown in FIG. 2.

In step 102, an insulating material 2 is prepared, and the metallic membrane 1 may be adhered on the insulating material 2. In the embodiment, the insulating material 2 may be paper, timber, Acrylonitrile-Butadiene-Styrene (ABS), polycarbonate (PC), polyethylene terephthalate (PET), polyimide (PI), thermoplastic elastomer or polylactic acid plastic.

In step 104, a photolithography process is performed. A mask resist layer 10 is formed on the metallic membrane 1, and exposed by a stepper (not shown in drawings) to form an exposed film 101 having a specific circuit (as shown in FIG. 3) through the reaction of resist layer 10. Next, a development process is performed by a developing machine. An unexposed resist layer 10 is dissolved, as shown in FIG. 4.

In step 106, an etching process is performed. The exposed portion of the metallic membrane 1 is etched by light or chemical reagent, as shown in FIG. 5. Next, the residual resist (the exposed film 101) is stripped in a stripper, and a metallic circuit layer 1a is formed on the insulating material 2, as shown in FIGS. 6 and 7. In the embodiment, the metallic circuit layer 1a includes an electrical connector 11a, a metallic circuit 12a connecting to the electrical connector 11a and an electrode section 13a connecting to an end of the metallic circuit 12a.

In step 108, a film plating process is performed. a semi-product completed by step 106 is plated at least one metallic film 1b on the metallic circuit layer 1a in an electroplating tank to form a protective layer, as shown in FIG. 8. A circuit layer 20 includes the metallic circuit layer 1a and the metallic film 1b. In the embodiment, the metallic film 1b may be conductive metallic material selected from the group consisting of palladium, nickel, platinum, tungsten, gold, titanium, copper, zinc, iridium, vanadium and zirconium.

In step 110, a sensing reagent is applied on the test strip. The amount and species of the sensing reagent are selected as required. The sensing reagent is applied on the electrode section 13a covered with the metallic film 1b to form a sensing reagent layer 3 by spray marking, as shown in FIG. 9.

In step 112, an intermediate layer 4 is adhered to the insulating material 2. After adhering, the intermediate layer 4 only covers the metallic circuit 12a and the electrode section 13a covered with the metallic film 1b of the circuit layer 20, and the electrical connector 11a is exposed. Also, the intermediate layer 4 has an opening 41 to expose the sensing reagent layer 3 and the electrode section 13a, as shown in FIG. 10. In the embodiment, the intermediate layer 4 is shorter than the insulating material 2. The intermediate layer 4 is of insulating material.

In step 114, a cover 5 is covered on the insulating material 2 and the intermediate layer 4. After the cover 5 is covered, the test strip only exposes the electrical connector 11a covered with the metallic film 1b. The cover 5 has two holes 51 corresponding to the opening 41. The two holes 51 are provided for user to instill biological fluid, as shown in FIG. 11. In the embodiment, the cover 5 is shorter than the insulating material 2. The cover 5 is of insulating material.

In step 116, the insulating material 2 is cut to form a plurality of insulating sheets 2a. Each of the insulating sheets 2a has the circuit layer 20, the sensing reagent layer 3, the intermediate layer 4 and the cover 5, as shown in FIGS. 12 and 13.

The test strip is determined after the fabrication has been completed. The resistance of the circuit layer 20 has a range from 0.01Ω to 2k Ω. The thickness of the circuit layer 20 has a range from 0.02 um to 30 um.

Also, as blood flows into inside of the test strip through the two holes 5, and the sensing reagent layer 3 sensings the blood, the resistance can keep stable during signal transmission because the circuit layer 20 is fabricated by adhering manner. Therefore, it can make glucose reaction much stable so that glucose meter can read accurate readings.

Please refer to FIGS. 12 and 13. FIG. 12 is a schematic top view of test strip for measuring biological fluid of the present invention. FIG. 13 is a schematic cross-sectional view of test strip for measuring biological fluid of the present invention. As shown in FIGS. 12 and 13, the test strip for measuring biological fluid of the present invention comprises an insulating sheet 2a, a circuit layer 20, a sensing reagent layer 3, an intermediate layer 4 and a cover 5.

The insulating sheet 2a may be paper, timber, Acrylonitrile-Butadiene-Styrene (ABS), polycarbonate (PC), polyethylene terephthalate (PET), polyimide (PI), thermoplastic elastomer or polylactic acid plastic.

The circuit layer 20 may be disposed on the insulating sheet 2a. The circuit layer 20 may include a metallic circuit layer 1a and a metallic film 1b disposed on the metallic circuit layer 1a. The metallic circuit layer 1a includes an electrical connector 11a, a metallic circuit 12a connecting to the electrical connector 11a and an electrode section 13a connecting to an end of the metallic circuit 12a. In the embodiment, the metallic circuit layer 1a and the metallic film 1b may be conductive metallic material selected from the group consisting of palladium, nickel, platinum, tungsten, gold, titanium, copper, zinc, iridium, vanadium and zirconium.

The sensing reagent layer 3 is applied on the electrode section 13a covered with the metallic film 1b. In the embodiment, the amount and species of the sensing reagent are selected as required.

The intermediate layer 4 covers the insulating sheet 2a and the metallic circuit 12a covered with the metallic film 1b of the circuit layer 20. Also, the intermediate layer 4 has an opening 41 to expose the sensing reagent layer 3 and the electrode section 13a covered with the metallic film 1b. In the embodiment, the intermediate layer 4 is shorter than the insulating sheet 2a. The intermediate layer 4 is of insulating material.

The cover 5 is covered on the insulating sheet 2a and the intermediate layer 4. After the cover 5 is covered, the test strip only exposes the electrical connector 11a covered with the metallic film 1b. The cover 5 has two holes 51 corresponding to the opening 41. The two holes 51 are provided for user to instill biological fluid. In the embodiment, the cover 5 is shorter than the insulating material 2. The cover 5 is of insulating material.

The test strip is determined after the fabrication has been completed. The resistance of the circuit layer 20 has a range from 0.01Ω to 2k Ω. The thickness of the circuit layer 20 has a range from 0.02 um to 30 um.

Also, as blood flows into inside of the test strip through the two holes 5, and the sensing reagent layer 3 senses the blood, the resistance can keep stable during signal transmission because the circuit layer 20 is fabricated by adhering manner. Therefore, it can make glucose reaction much stable so that glucose meter can read accurate readings.

Moreover, in case that the metallic circuit layer 1a have features of anti-oxidation and sensing, the step 108 of film plating may be omitted in the fabrication of the test strip, and thus the fabrication of the test strip is simple and save working hours.

While the invention is described in by way of examples and in terms of preferred embodiments, it is to be understood that the invention is not limited thereto. On the contrary, the aim is to cover all modifications, alternatives and equivalents falling within the spirit and scope of the invention as defined by the appended claims.

What is claimed is:

1. A method of fabricating a test strip for measuring biological fluid comprising steps of:
(a) preparing a metallic membrane and an insulating material;
(b) adhering the metallic membrane on the insulating material;
(c) photolithographing for the metallic membrane;
(d) etching the metallic membrane to form a circuit layer connected to an electrode section;
(e) providing a sensing reagent on the electrode section to form a sensing reagent layer;
(f) adhering an intermediate layer to the insulating material to cover the circuit layer and expose part of the circuit layer, the intermediate layer having an opening to expose the sensing reagent layer and the electrode section; and
(g) adhering a cover to cover the intermediate layer and the insulating material and expose part of the circuit layer, the cover having two holes corresponding to the opening.

2. The method of fabricating a test strip for measuring biological fluid according to claim 1, wherein the metallic membrane of step (a) is a conductive metallic material selected from the group consisting of palladium, nickel, platinum, tungsten, gold, titanium, copper, zinc, iridium, vanadium and zirconium.

3. The method of fabricating a test strip for measuring biological fluid according to claim 2, wherein the insulating material of step (b) is paper, timber, Acrylonitrile-Butadiene-Styrene (ABS), polycarbonate (PC), polyethylene terephthalate (PET), polyimide (PI), thermoplastic elastomer or polylactic acid plastic.

4. The method of fabricating a test strip for measuring biological fluid according to claim 3, wherein the photolithography process of step (c) is performed by forming a mask resist layer on the metallic membrane, and exposed by a stepper to form an exposed film through the reaction of resist layer, performing a development process to dissolve an unexposed resist layer.

5. The method of fabricating a test strip for measuring biological fluid according to claim 4, wherein the circuit layer of step (d) is formed by etching the exposed portion of the metallic membrane in an etching process, the residual resist is stripped, and a metallic circuit layer is formed on the insulating material.

6. The method of fabricating a test strip for measuring biological fluid according to claim 5, wherein the metallic circuit layer includes an electrical connector, a metallic circuit connecting to the electrical connector and an electrode section connecting to an end of the metallic circuit.

7. The method of fabricating a test strip for measuring biological fluid according to claim 6, wherein the sensing reagent of step (e) is applied on the electrode section to form a sensing reagent layer by spray marking

8. The method of fabricating a test strip for measuring biological fluid according to claim 7, wherein the intermediate layer of step (f) is adhered to the insulating material to cover the metallic circuit and the electrode section of the circuit layer and expose the electrical connector.

9. The method of fabricating a test strip for measuring biological fluid according to claim 8, wherein the intermediate layer is shorter than the insulating material.

10. The method of fabricating a test strip for measuring biological fluid according to claim 9, wherein the intermediate layer is of insulating material.

11. The method of fabricating a test strip for measuring biological fluid according to claim 10, wherein step (g) after the cover is covered, the test strip only exposes the electrical connector covered with the circuit layer, and the cover is shorter than the insulating material.

12. The method of fabricating a test strip for measuring biological fluid according to claim 11, wherein the cover is of insulating material.

13. The method of fabricating a test strip for measuring biological fluid according to claim 9, after step (g) further comprising the insulating material is cut to form a plurality of insulating sheets, each of the insulating sheets has the circuit layer, the sensing reagent layer, the intermediate layer and the cover.

14. The method of fabricating a test strip for measuring biological fluid according to claim 13, between steps (d) and (e) further comprising a metallic film is plated on the metallic circuit layer.

15. The method of fabricating a test strip for measuring biological fluid according to claim 14, wherein the metallic film is a conductive metallic material selected from the group consisting of palladium, nickel, platinum, tungsten, gold, titanium, copper, zinc, iridium, vanadium and zirconium.

16. The method of fabricating a test strip for measuring biological fluid according to claim 15, wherein the resistance of the circuit layer has a range from 0.01Ω to 2k Ω.

17. The method of fabricating a test strip for measuring biological fluid according to claim 16, wherein the thickness of the circuit layer has a range from 0.02 um to 30 um.
